(19) United States
(12) Patent Application Publication
(10) Pub. No.: US 2006/0276721 A1
(43) Pub. Date: Dec. 7, 2006

(54) Pedicle impedence measuring probe

(76) Inventor: William J. McGinnis, Cincinnati, OH (US)

Correspondence Address: LADAS & PARRY LLP, 224 SOUTH MICHIGAN AVENUE, SUITE 1600, CHICAGO, IL, 60604, US

(21) Appl. No.: 11/378,177

(22) Filed: Mar. 16, 2006

Related U.S. Application Data
(63) Continuation-in-part of application No. 11/292,861, filed on Dec. 2, 2005.
(63) Continuation-in-part of application No. 11/144,214, filed on Jun. 3, 2005.

(51) Int. Cl. A61B 5/05

(52) U.S. Cl. 600/547

(57) ABSTRACT

A probe, a system and method for monitoring correct placement of a pedicle screw in a vertebral body dynamically measures impedance on either side of a pedicle screw during dynamic pedicle screw placement in a pedicle bone structure. Dynamic impedance is also assessed in real-time.

### CROSS REFERENCE TO RELATED APPLICATIONS

This application is a continuation-in-part of application Ser. No. 11/292,861 filed Dec. 2 2005, and Ser. No. 11/144,214 filed Jun. 3 2005, which are incorporated by reference herein in their entirety.

### TECHNICAL FIELD

This invention relates to the field of intraoperative neurophysiology, and more particularly to the intraoperative placement of pedicle screws during procedures for lateral fixation of the lumbar-sacral spine.

### BACKGROUND

Posterior spinal fusions with lateral fixation of adjacent vertebral bodies with pedicle screws and rods is procedurally well-documented in the medical literature, and the use of pedicle screws for spinal stabilization is commonplace. However, placement of these screws is largely done blindly, and despite surgical inspection and imaging techniques, the incidence of improperly positioned pedicle screws and resulting neurological impairment has been reported to be high (Bose et al., Spine 2002). With recent attention turning to minimally invasive operative procedures, the risk of misplaced hardware is even greater since during minimally invasive operative procedures the small incision affords a limited view of the anatomical structures.

An abroachment of the pedicle structure would warrant a redirection of the screw to prevent clinical manifestation (pain, paralysis, hemorrhaging, etc.) and the need for further intervention. During these procedures the operative incision affords visual inspection of the pedicle for anatomical correlation.

Electrophysiological intraoperative monitoring techniques have been used to assess somatosensory and dermatomal nerve root function, and more recently techniques which rely on spontaneous and triggered myogenic responses recorded from muscles innervated by roots at risk. Triggered myogenic activity, performed by applying an electrical current to a pedicle feeler and evaluating triggered activity from innervated musculature is problematical. If a triggered response is not seen on lower stimulus intensities of up to 8-10 mA, but is then seen on intensities greater than 10 mA, the placement is regarded as being safe, but several factors can influence their accuracies including degree of muscle relaxation, current shunting, nerve root compression or disease.

Furthermore, problems with using literature derived thresholds for signal abroach arise due to different bone types presenting with different stimulus intensities to define safety. Moreover, different patients have different impedance to current flow which may skew assessments of efficacy of placement.

There remains therefore a need for a system for persistent assessment during pedicle screw placement that can provide reliable early warning of abroach when abroach is occurring, particularly during minimally invasive procedures.

It has been surprisingly found that individual assessment of each vertebral body's electrical impedance provides an efficient way of assessing a pedicle screw placement.

### SUMMARY OF THE INVENTION

The present invention addresses the problems outlined above by providing a pedicle impedance probe for measuring the impedance of a pedicle bone structure, comprising: a tube having a distal end, the tube adapted to house at least one medial impedance wire and at least one lateral impedance wire; at least one medial impedance wire extending to the distal end of the tube and terminating at a medial terminal, the medial terminal being attached to the distal end of the tube; and at least one lateral impedance wire extending to the distal end of the tube and terminating at a lateral terminal, the lateral terminal being attached to the distal end of the tube; such that the medial and lateral terminals are spaced at a fixed distance from each other and can be inserted into a pedicle bone structure.

In another view of the invention, the invention is also directed to a system comprising the probe of the invention in connection with an impedance monitor and optionally real-time monitoring and assessment capability.

In a further view of the invention, the invention is directed to a method for measuring impedance in a bony body, comprising: attaching the probe of claim 1 to a bony body; measuring a first impedance between the medial terminals and lateral terminals; and serially measuring subsequent impedances, wherein a change in impedance is indicative of a change in the integrity of the bony body.

It should be appreciated that the present invention can be implemented and utilized in numerous ways, including without limitation a device, an apparatus, a system, and a method for applications now known and later developed. These and other unique features of the system disclosed herein will become more readily apparent from the following description and the accompanying drawings.

### DESCRIPTION OF DRAWINGS

FIG. 1 is a perspective view of a pedicle impedance probe.

FIG. 2 shows an enlarged view of the probe distal end.

FIG. 3 shows a view of a pedicle, pedicle impedance probe and pedicle screw.

FIG. 4 shows an enlarged view of the probe distal end of an alternative embodiment.

FIGS. 5-10 show top views of probe alternative embodiments.

FIG. 11 illustrates a pedicle impedance monitoring system.

Like reference symbols in the various drawings indicate like elements.

### DETAILED DESCRIPTION

The invention provides a probe for assessing pedicle screw placement intraoperatively, particularly minimally invasive pedicle screw placement. The probe comprises a hollowed tube having wired needle attachments for tapping into the vertebra (posterior, lamina or pedicle?), through which hollowed tube pedicle screw placement is performed. The needle attachments are spaced apart such that they may be medially and laterally orientated with respect to placement of the tube, and measured impedance between the needles during dynamic screw placement represents impedance of bone between the needles. A change in impedance represents a breach of the pedicle wall and would indicate the need for dynamic redirection of the pedicle screw.

The invention is predicated on the following principles: different patients have different impedance to current flow for identifying abroachment; different bone types have different impedance; persistent assessment during screw placement (rather than following screw placement) provides early warning of abroachment; and individual assessment of each bony body's electrical impedance is an efficient way of assessment pedicle screw placement.

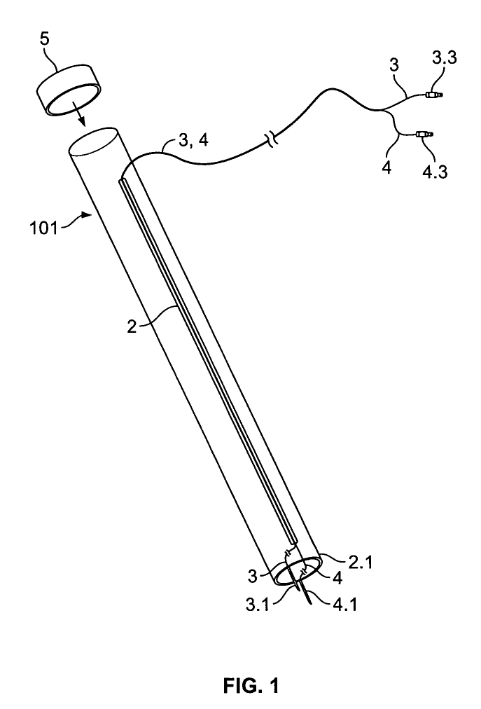

The FIGS. 1-6 show various embodiments of a pedicle impedance probe.

In FIG. 1, tube (101), that is preferably a clear plastic tube, has medial (3) and lateral (4) impedance monitoring wires running down the length (2) of the tube (101) and terminating at distal end (2.1) of tube (101) in, respectively, a medial terminal (3.1) and a lateral terminal (4.1). Medial (3) and lateral (4) impedance monitoring wires are connected or attached proximally to connectors (3.3) and (4.3) for ready connection to an impedance monitor (not shown). Optional cap (5) fits onto the top of the tube. By distal end of the tube is meant according to practice the bottom end of the tube and closest to the target bony body.

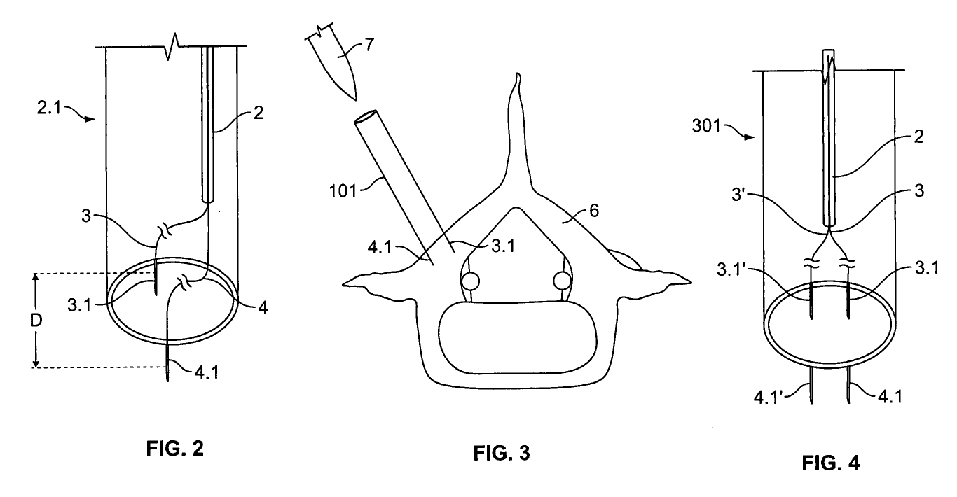

For impedance measurement, the medial (3.1) and lateral (4.1) terminals are spaced approximately 180° apart, and are separated from each other by a fixed and known distance (D) (see FIG. 2).

In the embodiment shown in FIG. 1, medial terminal (3.1) and lateral terminal (4.1) are needles which can be tapped down into the pedicle causing terminals (3.1), (4.1) to be in contact with the bone of the pedicle. Medial terminal (3.1) and lateral terminal (4.1) are preferably surgical grade needles which can be embedded in the pedicle bone structure. In a surgical procedure, a pedicle screw is passed down the tube (101) and is screwed into the pedicle. When connectors (3.3) and (4.3) are attached to an impedance monitor, the impedance between the two terminals (3.1), (4.1) can be measured before and during the screwing down of the pedicle screw into the pedicle.

FIG. 2 shows an enlargement of the probe distal end (2.1) in which medial (3) and lateral (4) impedance monitoring wires running down tube length (2) terminate in medial terminal (3.1) and lateral terminal (4.1) of a fixed known distance (D) apart.

The principle behind the invention is that impedance is measured between medial and lateral terminals (3.1), (4.1) to provide a measurement of the pedicle impedance which can indicate a change in the integrity of the pedicle. The pedicle impedance between the medial terminal and lateral terminal provides an impedance for the bone between the terminals at time of measurement. If a subsequent measurement shows an impedance change, such a change in the pedicle impedance can indicate pedicle integrity change and a likely abroachment.

The number and spacing of impedance monitoring wires is a design choice. Although FIG. 1 shows a single medial (3) wire and a single lateral (4) wire, more than a single medial wire and a single lateral wire may also be used in keeping with the spirit of the invention. For example, two medial terminal and two lateral terminals could also be used and the impedance between the pairs measured. Impedance measurements representing the impedance between the points medial and lateral of the position of the pedicle screw would similarly provide the given impedance for the bone between the terminals at time of measurement, and when subsequent measurements are taken between the terminals, an increase in impedance would indicate a change in the pedicle integrity and a likely abroachment.

FIG. 3 shows tube (101) in which medial (3) and (3′) lateral (4) and (4′) impedance monitoring wires running down tube length (201) terminate in medial terminals (3.1) and (3.1′) and lateral terminals (3.2) and (3.2′), the distance between medial (3) and lateral (4) impedance monitoring wires being of a fixed known distance apart, and likewise the distance between medial (3′) and lateral (4′) impedance monitoring wires being of a fixed known distance apart.

FIG. 3 shows tube (101) inserted into the pedicle (6) in preparation for a pedicle screw (7) to be passed down through tube (101) and screwed into place in the pedicle (6). Tube (101) is positioned such that medial terminal (3.1) is placed medial to the eventual position of the pedicle screw, and lateral terminal (4.1) is placed lateral to the eventual position of the pedicle screw, and such that impedance measurements in pedicle (6) across the implanted screw (7) can provide an early warning of abroachment of pedicle (6) by screw (7).

FIG. 4 shows an enlargement of the probe distal end (301) having two medial and two lateral impedance monitoring wires running down length (2) of the tube. In this figure, two medial impedance monitoring wires (3), (3′) terminate in two medial terminals (3.1), (3.1′) and two lateral impedance monitoring wires (not shown in the figure) terminate in two lateral terminals (4.1), (4.1′). Impedance is measured between the medial terminals (3.1), (3.1′) and lateral terminals (4.1), (4.1′). The distance (not shown in the figure) for the impedance measurement is taken as the space apart of a line between medial terminals (3.1) and (3.1′) from a line between lateral terminals (4.1), (4.1′).

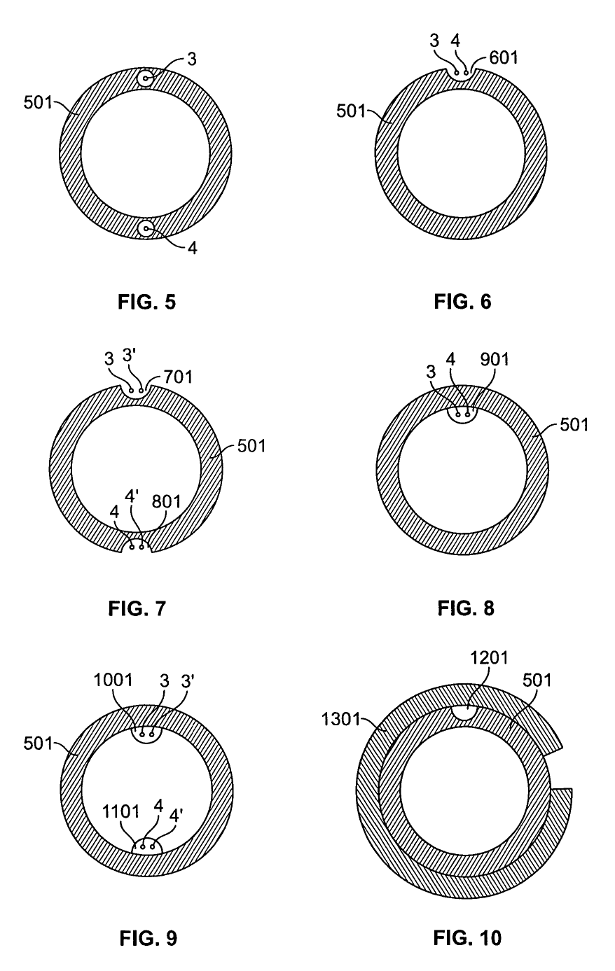

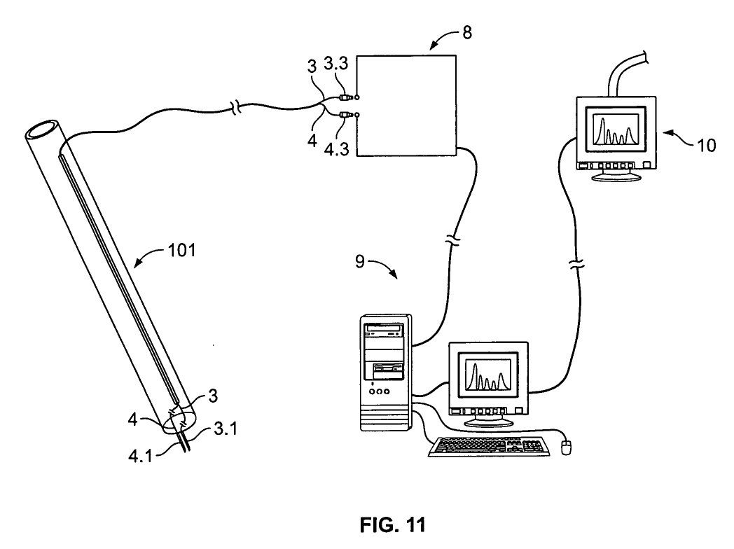

The medial and lateral impedance monitoring wires can be extended down the tube in various different ways which can be seen in FIGS. 5-10, all of which show a top view of the pedicle impedance probe. FIG. 5 shows top view of in which medial (3) and lateral (4) impedance monitoring wires are positioned on either side of the probe in, respectively, space (301) and (401) in tube wall thickness (501). FIG. 6 shows an embodiment in which medial (3) and lateral (4) impedance monitoring wires are positioned in close proximity in a cavity (601) in the outer tube wall. FIG. 7 shows an embodiment having two medial (3), (3′) and two lateral (4), (4′) impedance monitoring wires, positioned in outer tube wall cavities (701) and (801) respectively. FIG. 8 shows an embodiment in which the cavity (901) is situated in the inner wall of the tube having medial (3) and lateral (4) impedance monitoring wires are positioned in close proximity. FIG. 9 shows an embodiment in which cavities (1001) and (1101) in the inner wall of the tube house, respectively, medial (3) (3′) and lateral (4), (4′) impedance monitoring wires. FIG. 10 shows cavity (1201) for housing medial and lateral monitoring wires in tube wall (501) with outer sleeve (1301) adapted to slide round and expose cavity (1201).

FIG. 11 shows a system comprising a pedicle impedance probe having tube (101), having medial (3) and lateral (4) impedance monitoring wires terminating distally, respectively, in medial terminal (3.1) and a lateral terminal (4.1), and proximally connected to connectors (3.3), (4.4) respectively in impedance monitor (8). Impedance monitor (8) is connected via processor (9) to real-time monitoring device (10) for displaying measurements in real-time on a surgeon's display screen.

During a surgical procedure, when the pedicle in which the screw is being placed is cleared of soft tissue to expose the medial and lateral parts of the pedicle, the probe is visually orientated over the pedicle so that the medial impedance needle is approximately positioned on the medial side of the pedicle, and the lateral impedance needle is over the lateral side of the pedicle. Once the orientation of the tube over the pedicle is satisfactory, the needles are then tapped down into the bone. An impedance reading taken in the bone between the medial and lateral needles, the impedance representing the integrity of the bone between the needles. If the wall is broached, impedance between the two needles increases. Impedance is measured continuously throughout the drilling procedure.

In a preferred embodiment therefore, the probe is a reusable device comprising a clear plastic sterilizable hollow tube having impedance wires running down a longtitudinal cavity in the wall of the tube. The tube may be regarded as having a medial and a lateral side, the medial side being orientated to the medial side of the pedicle screw placement site, the lateral side being orientated to the lateral side of the placement site. In a procedure for pedicle screw placement, the medial side of the site for placement is the side adjacent to the nerve having an impedance wire running down a cavity on the medial side of the tube and an impedance wire running down a cavity in the tube on its lateral side.

In one preferred embodiment, the tube has two wire attachments positioned for orientating medially and laterally with respect to placement of the pedicle screw.

In another embodiment, the tube is made of a molded clear sterilizable plastic so that the surgeon is able to view the surgical field through the tube and see that the drill is going straight.

In a further embodiment, the internal diameter of the tube is a design choice, and is preferably between about 5 and 8 mm.

In a preferred embodiment the needles are gold plated, having sharp medical grade points, and being 13 mm, 14 guage, preferably having about 8 mm exposed at the distal end of the tube.

As can be seen in the embodiment shown in FIG. 1, the tube may be capped during the procedure. The cap may be of three sizes to fit tube top. At top of the tube, the impedances are fed via connectors I to an impedance meter, thence to computer and video out to surgeon's display screen.

In one preferred embodiment, at the top of the tube, two marks differentiate the two impedance wires leading to the respective needles, so that the needles may be correctly orientated in the pedicle body by the surgeon. The differentiating marks may be colored (for example red for the medial impedance and blue for the lateral impedance).

In an ideal embodiment, the two marks and the emerging two impedances attached to the needles at the distal end of the tube are 180 degrees apart.

Impedance is a measure of opposition to a sinusoidal electrical current, or the overall opposition to current presented by a circuit, expressed in ohms (Ω). The total impedance of a circuit is the square root of the sum of the squares of the resistance and reactance, √(R²)+(X²) where R is resistance and X is reactance. Impedance is directional. If a first input is medial, the vector of the loop will be medial to lateral. Any change would be known by looking at the direction of both impedances both displayed. An increase in the first-to-second loop impedance would indicate a medial broach.

What is claimed is:

1. An pedicle impedance probe for measuring the impedance of a pedicle bone structure, comprising:
a) a tube having a distal end, the tube adapted to house at least one medial impedance wire and at least one lateral impedance wire;
b) at least one medial impedance wire extending to the distal end of the tube and terminating at a medial terminal, the medial terminal being attached to the distal end of the tube; and
c) at least one lateral impedance wire extending to the distal end of the tube and terminating at a lateral terminal, the lateral terminal being attached to the distal end of the tube; such that the medial and lateral terminals are spaced at a fixed distance from each other and can be inserted into a pedicle bone structure.

2. The probe of claim 1, wherein the medial and lateral terminals are spaced approximately 180° apart from each other on a line passing through the tube's central axis.

3. The probe of claim 1, comprising a single medial impedance wire and a single lateral impedance wire.

4. The probe of claim 1, comprising a pair of medial terminals and a pair of lateral terminals.

5. The probe of claim 1, wherein the medial and lateral terminals are needles.

6. The probe of claim 1, wherein the medial and lateral wires are connected to an impedance monitor.

7. The probe of claim 1, wherein the tube is transparent.

8. The probe of claim 7, wherein the tube is made of a sterilizable plastic.

9. The probe of claim 1, wherein the terminals are adapted to communicate wirelessly with an impedance monitor.

10. The probe of claim 1, wherein the impedances are comprised of gold.

11. A system comprising the probe of claim 1 in connection with an impedance monitor.

12. The system of claim 11, further comprising a computer in connection with the monitoring device, the computer having the capability for performing real-time monitoring and assessment.

13. A method for measuring impedance in a bony body, comprising:
a) attaching the probe of claim 1 to a bony body;
b) measuring a first impedance between the medial terminals and lateral terminals; and
c) serially measuring subsequent impedances, wherein a change in impedance is indicative of a change in the integrity of the bony body.

14. The method of claim 13, wherein the bony body is a pedicle.

15. The method of claim 13, wherein step a) further comprises inserting a pedicle screw into the pedicle through the probe and then measuring a first impedance.

16. The method of claim 13, wherein in step c) the serial measurements are made after the pedicle screw is inserted into the pedicle, and a change in impedance is indicative of an abroachment of the pedicle.

17. The method of claim 13, further comprising step d) monitoring and assessing impedance in real-time.
